(19) United States
(12) Patent Application Publication
(10) Pub. No.: US 2011/0287508 A1
(43) Pub. Date: Nov. 24, 2011

(54) BIOREACTORS FOR TISSUE ENGINEERING

(76) Inventors: Julian B. Chaudhuri, Somerset (GB); Marcus Jarman-Smith, Somerset (GB); Karina Stewart, Somerset (GB)

(21) Appl. No.: 12/678,849

(22) PCT Filed: Sep. 19, 2008
(86) PCT No.: PCT/GB2008/003172

§ 371 Date: Aug. 12, 2010

(30) Foreign Application Priority Data
Sep. 19, 2007 (GB) 0718245.4

(51) Int. Cl. C12N 11/00; C12M 3/00

(52) U.S. Cl. 435/174; 435/289.1

(57) ABSTRACT

Bioreactors and methods of using them to produce tissue engineered products or culture cells are disclosed, and more particularly on the development of a tissue and cell culture method based upon an expanded bed bioreactor in which an initial resting bed of particles on which or in which cells are attached, encapsulated or immobilised have a fluid passed upwards through the bed to form an expanded bed in which the fluid acts to separate the particles, i.e. under plug flow conditions to enable the relative positions of the particles to be maintained during the step of culturing the cells to form tissue and helps to reduce collisions between particles and turbulent flow or convective mixing.

### FIELD OF THE INVENTION

The present invention relates to bioreactors for tissue engineering, and more particularly to expanded bed bioreactors and methods of using the bioreactors for making tissue engineered products.

### BACKGROUND OF THE INVENTION

A range of techniques are known in the art in which particulate beds are employed for culturing cells expressing useful bioproducts, such as recombinant proteins, and for separating the bioproducts thus produced. Some of these techniques involve passing fluid upwards through a bed of particles, known in the art as fluidized beds. In fluidized beds, the velocity of the fluid through the bed of particles is normally sufficient to cause turbulent motion of the particles to assist in mixing and contacts between the particles and the fluid medium.

An expanded bed is a particular subset of the wider class of fluidized beds. An expanded bed is characterised by the lack of particle-particle interactions and minimal back-mixing. The expanded bed concept was originally developed for application as a chromatography process for the recovery and purification of genetically engineered proteins in biotechnology. Mainly known as “expanded bed adsorption” (EBA) this technique has found quite good acceptance in the biotechnology industry. Briefly, EBA integrates solid-liquid separation and a first chromatography step. Suitable chromatography adsorbent particles are expanded by the upflow of the contents of the fermenter and contain the soluble target protein. This protein adsorbs to the particles thus recovering it from the feedstock. The protein-loaded particles are then packed down into a fixed bed and the protein recovered from the beads by washing with a suitable buffer. EBA has been used in the past for protein purification, and examples of its application to protein purification include glucose-6-phosphate dehydrogenase (G6PDH), B-phycoerythrin and clotting factor IX. A key to expanded bed adsorption is the properties of the adsorbent particles. Factors such as particle material, size and density have been well studied. In addition, there have been many fundamental studies on fluid dynamics and fluid flow in expanded bed protein purification.

A different type of fluidized bed bioreactor has been employed for culturing mammalian cells in bioprocessing, where the air used to oxygenate the cells is used to move the cells suspended in the liquid phase. In this case, cells are freely suspended, or are attached to microcarriers and suspended, in a liquid media and mixing is provided by an upward flow of air. Typically, the bioreactor is cylindrical in shape and the airflow is introduced at the bottom of the reactor. Mixing is provided by the rising air bubbles, which have a lower density than the surrounding fluid. This creates a volume of the fluid that has a lower effective density than the bulk fluid and thus moves upwards. At the top of the reactor, the gas disengages and the effective liquid density is now greater than the liquid at the top of the reactor and thus descends to the bottom of the reactor. This process sets up a circulation loop driven by the upward airflow and changing density of the fluid.

While these airlift bioreactors have sometimes been used to grow mammalian cells that have been genetically engineered to secrete pharmaceutical proteins into the media, airlift bioreactors are generally not used any more in large-scale mammalian cell culture.

The same bioreactor principle can also be used for the culture of yeast or bacterial cells, again freely suspended or immobilised particles, and for biocatalysis using immobilised enzymes. Recent applications involving non-mammalian cells and enzyme-catalysed reactions include cyclodextrin glucanotransferase (CGTase) production by alginate-immobilized cells, biodegradation of phenol, glucoamylase production with immobilised Kluyveromyces lactis and bioremediation of phenolic waste waters.

The repair of tissue in vivo can, in principle, be achieved using viable tissue implants that have been cultured in vitro. The manufacture of tissue constructs relies to a large degree on the use of bioreactor systems that can provide a controlled, in vivo-like environment for cells to proliferate on biodegradable matrices to form nascent tissues. Such reactors are generally operated such that the cells are fed by the flow (perfusion) of media through the cells or across the cells' surface. Because of poor diffusion within the scaffolds, the cells within the tissue constructs become starved of nutrients and do not proliferate well or even die. It is worth noting that in vivo, cells within tissue lie no more than 100-200 μm from a capillary and thus a source of nutrient. Such dimensions will need to be reproduced, if possible, in any in vitro culture system to prevent nutrient limitation.

In view of this and other problems in the art, the commercial manufacture of tissue is currently limited to planar products (e.g. dermal replacements) of less than 1 mm in thickness or to small particulate products (e.g. for articular cartilage replacement). A challenge for the tissue engineer has, to date, arisen from the need to provide tissues for certain key applications, e.g. replacement of whole meniscal cartilage in the knee, or replacement of thick sections of the osteochondral plate, where the dimensions are of the order of several millimetres in the smallest direction. Under these circumstances it is difficult to ensure nutrition of the tissue construct by using forced perfusion because of the small and diminishing permeability of the maturing construct during culture. If reliance is placed instead upon convective or diffusive mixing within the construct (e.g. the suspension of the construct within a stirred vessel of culture medium), then the outer layers of neo-tissue are adequately supplied with nutrients at the expense of the inner.

In addition, there is a need to efficiently culture progenitor cells (“stem cells”) such that the numbers of these rare cells can be increased to give clinically-relevant numbers whilst maintaining their ability (pluripotency and/or multipotency) to differentiate to other cell types without actually differentiating, that may be subsequently used in therapy. Furthermore, there is a need to be able to differentiate the progenitor cells effectively to give differentiated, terminal cell types that may be used in therapy, or for tissue engineering.

Accordingly, there is a continuing need in the art for improvements to methods and bioreactors for making tissue engineered products and culturing progenitor cells.

### SUMMARY OF THE INVENTION

Broadly, the present invention is based on a bioreactor and method of using it to produce tissue engineered products or culture cells, and more particularly on the development of a tissue and cell culture method based upon an expanded bed bioreactor in which an initial resting bed of particles on which or in which cells are attached, encapsulated or immobilised have a fluid passed upwards through the bed to form an expanded bed in which the fluid acts to separate the particles. The present invention is based on the realisation that forming an expanded bed of the particles under plug flow conditions helps to enable the relative positions of the particles to be maintained during the step of culturing the cells to form tissue and helps to reduce collisions between particles and turbulent flow or convective mixing. These latter processes are detrimental to the production of viable tissue engineered products or cells, and especially three dimensional tissue engineered products. In this method, conveniently the fluid used is the medium used for culturing the cells which can be pumped into a bottom portion of the bioreactor to cause the expanded bed to form. The passage of fluid or media through the bed also enables nutrients and other materials to be made available to the cells on the particles, helping to reduce the problem in the prior art of supplying the inner regions of tissue constructs with nutrients.

Thus, the bioreactors used in accordance with the present invention generally operate in two discrete modes. Firstly, in an expanded bed mode, the bioreactor allows the growth and proliferation of cells, including undifferentiated progenitor cells, on the particles with the direction of fluid flow from the bottom of the bioreactor to the top. After a suitable period of culture, where progenitor cells are used, they may be differentiated to terminal cell types by addition of growth factors, specific media components or other chemicals used for cell differentiation. Secondly, by stopping or reversing the flow of the fluid or media (i.e. so that there is no flow or top to bottom flow), the particles on which or in which the nascent tissue elements have started to form can be brought into contact, compressed if necessary, and the tissue elements directed to self-assemble into larger, complex three dimensional constructs, including tissue engineered products that have three dimensional geometries, as opposed to being planar or particulate. In this phase, any progenitor cells may be differentiated to terminal cell types by addition of growth factors, specific media components or other chemicals used for cell differentiation, and then allowed to progress to tissue formation. The process of producing the tissue engineered product may entail further culturing of the cells to facilitate the assembly of the tissue elements.

The principle underpinning the expanded bed bioreactor relies upon the ability of an expanded column of liquid medium to support a particulate bed in floatation provided that certain criteria are met. These criteria preferably include the following. Firstly, it is preferred that the column of fluid or medium possesses a minimal variation in axial velocity across the cross-section of the column to help to eliminate back mixing. For a cylindrical column in which fluid or media is pumped upwards axially through the bed of cells and particles in the cylinder, the minimal variation will be across the radius of the cylinder.

Secondly, the particles must be buoyant at the moderate superficial velocities required to form the expanded bed, by virtue of factors such as their density and exposed surface area to the flowing medium. If the balance is suitable, then form drag will equal gravitational force for each particle under the chosen conditions and the particle remains suspended in the flow chamber of the bioreactor with negligible movement.

Thirdly, the polydispersity of the particle properties must satisfy conditions that provide a range of rest positions within one batch, i.e. larger particles tend to settle down near the bottom part of the expanded bed and smaller ones near the top, resulting in a satisfactory spectrum of rest positions for the particles and hence a bed of particles which are relatively immobile in suspension and oscillate about some steady position. By way of example, in the present invention, the population of particles preferably have an average particle diameter than may vary from 50 um to 5 mm depending on the material used for fabrication and the particle structure.

If the above conditions are met it becomes possible to support a bed of particles in a volume typically about 0.75 to 4.0, times and more preferably about 1.5 to 3.5, the rest (packed) volume, and more specifically about three times the rest (packed) volume. During expansion the position of each particle is relatively stationary with respect to its nearest neighbours and collision frequencies are low. The resultant system operates under plug flow at moderate fluid shear. Such conditions will be attractive to cell growth whilst minimising diffusion limitations. In this connection, it is important to note that the bioreactor configuration is not a “fluidised bed”, as fluidised beds are highly turbulent, operate at very high rates of fluid throughput and are characterised by very high values of fluid shear and high frequencies of particle-particle collision.

Once the cell growth has been sufficiently established the bioreactor may be operated in the compression phase. The expansion will be stopped and the cells allowed to settle under gravity, or the flow reversed, putting the cells into a compact three dimensional bed, mimicking the complex structure of the required tissue. The result should be a tissue construct where the cells at the core will be proliferating at about the same rate as those on the surface.

Accordingly, in a first aspect, the present invention provides a method of producing a tissue engineered product in a bioreactor which comprises:
(a) immobilising or encapsulating cells for forming the tissue engineered product on or in particles of a scaffold material;
(b) forming a packed bed of the particles on which the cells are immobilised or encapsulated in a flow chamber of the bioreactor;
(c) passing cell culture media through the bed of particles in the flow chamber at a velocity sufficient to separate the particles to form an expanded bed under conditions tending towards plug flow and which substantially maintains the relative positions of the particles in the flow chamber;
(d) culturing the cells so that they proliferate on the particles to begin to form tissue elements; and
(e) forming the tissue elements on the particles to produce the tissue engineered product.

Preferably, the step (e) of forming the tissue engineered product comprises promoting interactions between the cells on or in the particles, for example to encourage the formation of the extracellular matrix. Conveniently, this may be achieved by stopping or reducing the flow of the fluid, thereby allowing the particles to settle under gravity so that the nascent tissue elements assemble to form the tissue engineered product. Alternatively or additionally, the flow of fluid may be reversed to encourage the particles to produce matrix molecules and form the tissue engineered product. In either case, the method may then comprise a further step of culturing the cells to encourage assembly of the tissue elements into a finished tissue engineered product. Where this approach is used, the present invention has the advantage that the growth and proliferation of the cells has been started under the favourable conditions of the expanded bed in which they are well supplied with nutrients and this growth and proliferation continues when the cells forming the tissues elements are assembled into a tissue engineered product.

In a further related aspect, the present invention provides a method of culturing progenitor cells in a bioreactor which comprises:
(a) immobilising or encapsulating the progenitor cells on or in particles of a scaffold material;
(b) forming a packed bed of the particles on which the cells are immobilised or encapsulated in a flow chamber of the bioreactor;
(c) passing cell culture media through the bed of particles in the flow chamber at a velocity sufficient to separate the particles to form an expanded bed under conditions tending towards plug flow and which substantially maintain the relative positions of the particles in the flow chamber;
(d) culturing the progenitor cells so that they proliferate on the particles; and
(e) optionally isolating the cultured cells.

In the aspects of the present invention, the cells are mammalian primary cells, progenitor cells or genetically modified cells. In embodiments of the invention in which the cells are progenitor cells, the method preferably comprises culturing the progenitor cells in the expanded bed phase thereby maintaining pluripotency and/or multipotency and differentiating the cells in either the expanded bed and/or compressed bed phase. Conveniently, the step of differentiating the progenitor cells comprises contacting the cells with growth factors, cytokines or other agents for differentiating progenitor cells to terminal cell types. More generally, culture media comprising nutrients and/or growth factors can be used to deliver these materials to the cells. The skilled person can readily chose nutrients and growth factors appropriate for different types of cultured cells based on the general knowledge in the art. By way of example the growth factors may include PDGF-AB, PDGF-BB, TGF-β1 and/or IGF-I. As used herein, the term “progenitor cells” includes stem cells. The cells are preferably mammalian in origin and are preferably human cells. In certain circumstances, it may be preferred to obtain cells for use in the method from the individual who is to receive the tissue engineered product.

As will be apparent from the discussion above, a preferred feature of the present invention is that it facilitates the production of three dimensional tissue engineered products that are otherwise difficult or impossible to produce using prior art techniques in which cells on the surface of the product are perfused with media. In this connection, preferably a three dimensional tissue engineered product has a smallest dimension that is typically at least 1 mm, and more preferably is at least 2 mm, more preferably at least 5 mm, and most preferably is at least 10 mm, for example where the product is non-particulate product and is, for example, generally planar. Examples of tissue engineered products that can be made using the bioreactors and methods of the present invention include articular or meniscal cartilage, bone tissue, ligament, tendon, nerve cells, liver, pancreas, cardiac and vascular tissue, cornea, adipose tissue, genito-urinary tissue and dental tissue. Also, the culture of progenitor cells, and the co-culture of more than one cell type in the same reactor, may be carried out in accordance with the present invention.

The particulate scaffolds that can be used include commercial microcarrier beads, natural polymers (including collagen, silk, gelatin, alginate, chitin, chitosan, fibrin, peptide hydrogels), modified natural polymers (e.g. PEGylated) and synthetic polymers (poly(lactic-co-glycolic-acid), poly(lactic acid), poly(glycolic acid), polycaprolactone, expanded polyurethante, polytetrofluorethylene), modifications of synthetic polymers, combinations of both natural and synthetic polymers. In general, it is preferred that the particles have an average diameter between about 50 um and about 5 mm, and may, for example, be formed from a biodegradable material. Depending on the application for which the method is used, the particles are porous or non-porous microspheres. Preferred porous particles have pore sizes between about 0.5 um and about 500 um. Alternatively or additionally, the particles may be modified to facilitate the attachment or immobilisation of cells on the surface or inside the particles, or to promote the growth or differentiation of the cells during the course of the method. By way of example, the particles are coated with a growth factor such as fibronectin.

In some embodiments, the methods of the present invention may comprise one or more additional steps. These steps include, inter alia, one or more of (i) culturing the progenitor cells or cells forming a tissue engineered product to facilitate assembly of the tissue elements, and/or (ii) removing the tissue engineered product from the bioreactor, and/or (iii) preserving and packaging the product and/or implanting the tissue engineered product in a patient in need of the product.

In a further aspect, the present invention provides a tissue engineered product produced according to the methods as described herein.

In a further aspect, the present invention provide a tissue engineered product or cells produced according to the methods as described herein for use in treating an individual in need of implantation of the tissue engineered product or cells.

In a further aspect, the present invention provides an apparatus for use in the methods disclosed herein. In one embodiment, the apparatus comprises:
a bioreactor comprising a generally cylindrical flow chamber having a fluid inlet and a fluid outlet, wherein the flow chamber receives a bed of particles of a scaffold material on which cells are immobilised or encapsulated;
an inlet flow adapter in fluid communication with the fluid inlet of the bioreactor chamber for providing an even flow distribution;
an upper adapter in fluid communication with the fluid outlet to prevent cell loss and to allow flow reversal and bed compression; and
a pump for circulating culture media through the chamber via the flow adapters;
wherein in use (a) cell culture media is passed through the bed of particles in the bioreactor from the inlet to the outlet at a velocity sufficient to separate the particles to form an expanded bed under plug flow conditions which substantially maintains the relative positions of the particles, (b) the cells are cultured so that they proliferate on the particles, and optionally start to form tissue elements, and (c) the flow of culture media is stopped or reversed to compress the tissue elements on the particles to produce the tissue engineered product.

While the flow chamber is generally cylindrical, the dimensions of the cylinder will be dependent on the scale of the apparatus. In the case of smaller or laboratory scale devices the flow will be along the long axis of the cylinder. However, industrial scale columns tend to be short and fat, i.e. the scale up is typically done by keeping the bed height the same (say 100 mm) but making the column much wider (say 100 cm as opposed to 1 cm in the lab). However, it is preferred the cylindrical bioreactor chamber may vary in diameter from 10 mm to greater than 100 cm depending on the scale up of the process to manufacturing.

Embodiments of the present invention will now be described by way of example and not limitation with reference to the accompanying figures.

### BRIEF DESCRIPTION OF THE FIGURES

FIG. 1. Schematic diagram of bioreactor system.

FIG. 2. Diagram showing the bioreactor system used in experiment 1.

FIG. 3. Diagram showing the bioreactor system used in experiment 2.

FIG. 4. Cell Attachment on Cytodex 1 after 14 and 19 days in bioreactor cultures. During expansion (14 days): no ascorbate (a) and with ascorbate (b). During compression (19 days): no ascorbate (c) and with ascorbate (d). Figures (a) and (b) haematoxylin stained. Figures (c) and (d) correspond to unstained samples.

FIG. 5. Cell Viability on Cytodex 1 after 14 days in culture. In rod-stirred controls: no ascorbate in the CM (a) and with ascorbate (b). In bioreactors on expansion phase: no ascorbate (c) and with ascorbate (d)

FIG. 6. Collagen I Expression of OMCs cultured for 21 days on Cytodex 1 in Bioreactor Cultures stained with FITC fluorescence and counterstained with DAPI (blue). In Bioreactors on compression phase: no ascorbate (a) DAPI stain and (b) FITC stain; and with ascorbate (c) DAPI stain and (d) FITC stain.

FIG. 7. Cell Metabolites in controls and bioreactor cultures (exp. 1): Glucose (a), Lactose (b), Ammonia (c).

FIG. 8. OMCs attachment on Cytodex 3 in bioreactor cultures after 14 days. Media supplemented with ascorbate (a); media supplemented with ascorbate and PDGF-AB (b).

FIG. 9. Cell Viability on Cytodex 1 and Cytodex 3 after 14 days in culture. Bioreactors on compression: OMCs grown on cytodex 3 with ascorbate in the CM (a) ascorbate and PDGF-AB (b), ascorbate and TGF-β1 (c). OMCs grown on cytodex 1 with ascorbate and PDGF-AB in the CM (d), ascorbate and TGF-β1 (e).

FIG. 10. OMCs Proliferation of OMCs grown on Cytodex 1 and Cytodex 3 in rod-stirred flasks and Bioreactor systems (DNA Assay). Arrow indicates the change from expanded bed to a packed bed in bioreactors.

FIG. 11. OMCs Proliferation of OMCs grown on Cytodex 1 and Cytodex 3 in rod-stirred flasks and Bioreactor systems (MTT Assay).

FIG. 12. Collagen I Expression of OMCs cultured for 14 days on Cytodex 3 in Bioreactor Culture supplemented with ascorbate and PDGF-AB. In Bioreactors on compression phase (a) DAPI stain and (b) FITC stain.

FIG. 13. GAG and Total Collagen Expression of OMCs cultured for 14 days on Cytodex 3 in Bioreactor Culture supplemented with ascorbate and PDGF-AB. In Bioreactors on compression phase (a) Safranin O/fast green b) Masson-Goldner Trichrome stain.

FIG. 14. Cell Metabolites in rod-stirred controls and Bioreactor System (experiment 2): Glucose (a), Lactose (b), Ammonia (c). Arrow indicates the change from expanded bed to a packed bed in bioreactors.

FIG. 15. Alginate/chitosan capsule bed expansion A) shows measured bed expansion B) shows the calculated percentage of expansion from resting state.

FIG. 16. Cytodex 3 microcarrier bed expansion A) shows measured bed expansion B) shows the calculated percentage of expansion from resting state.

FIG. 17. Particle bed expansion of alginate/chitosan capsules in a 15 mm diameter column.

FIG. 18. Particle bed expansion of Cytodex 3 microcarriers in a 15 mm diameter column.

FIG. 19. A) Calculated Reynolds values B) calculated drag/shear forces for an alginate/chitosan capsule as part of an expanded bed within a 15 mm diameter column.

FIG. 20. A) Calculated Reynolds values B) calculated drag/shear forces for a Cytodex 3 microcarrier as part of an expanded bed within a 15 mm diameter column.

### DETAILED DESCRIPTION

### Theory of Fluidization and Expanded Beds

The general principles for the operation of a fluidized bed can be understood from the example of a porous bed of solid spherical particles at the bottom of a cylindrical column. When spherical particles are packed in a column, the beads sit close together in contact with each other and leave little room for solids and liquids to pass through at any reasonable velocity. If a liquid is passed through this bed of particles from the bottom to the top, the particles do not move, and the liquid passes through the bed via the small tortuous channels, losing energy and creating a pressure drop across the bed of particles, i.e. the difference in pressure measured at the entrance and exit of the packed bed. If the liquid velocity, i.e. the volumetric flow rate divided by cross sectional area of the column, is then steadily increased a point occurs when the particles are no longer stationary and start to move or are fluidised by the upward movement of the liquid (McCabe et al, 1976).

As the liquid velocity is steadily increased, the pressure drop in the liquid across the packed bed length increases linearly. As the velocity increases, there comes a point when the pressure drop equals the force of gravity on the particles and they start to move.

Initially, the bed starts to expand slightly with the particles still in contact. The porosity, that is the percentage or fraction of the bed that is not filled with solid particles, of the bed increases and the pressure drop increases more slowly. As the liquid velocity is further increased the bed expands further, the porosity increases but the particles are still just in contact. Further increases in velocity cause the particles to separate from each other and fluidisation proper begins. Still further increases in velocity result in the particles moving more and more vigorously, moving in random directions and bumping into each other (McCabe et al., 1976).

Expanded beds exist in a small region of operating conditions (pressure drop and fluid velocity) that are intermediate between a packed bed in which there is substantially no particle movement and a fully fluidized bed where there is turbulent motion of the particles. Understanding and controlling the degree of expansion of the bed is essential in forming an expanded bed. Bed expansion results from a balance of the particle properties (buoyancy, drag) and fluid properties (density, viscosity, superficial velocity). These properties can be formulated into equations that can calculate the minimum and maximum fluid velocities required to create and maintain a stable expanded bed (Sonnenfeld et al., 2007). The Richardson-Zaki equation correlates the voidage of an expanded bed (ε) with the superficial liquid velocity (U) by two parameters: the terminal settling velocity of the particle (Uₜ) and the expansion index (n) (Richardson and Zaki, 1954).

U=Uₜεⁿ

In addition, the Richardson-Zaki equation can be used to predict the fluid velocity required to achieve a desired expanded bed height (Yates, 1983, Lin et al, 2006).

$$E = \frac{H}{{H}_{0 }} = \frac{1 - {ɛ}_{0}}{1 - ɛ}$$

where the expansion factor (E) represents the ratio of expanded bed height (H) to sedimented bed height (H₀).

A key characteristic of an expanded bed is the degree of mixing inside the bed. In a true expanded bed, the degree of back mixing must be minimal; the fluid flow pattern from the bottom to the top should be as close as possible to plug flow (Chase & Draeger, 1992). Thus, as used in the present invention, “an expanded bed” includes a stable fluidized bed where the substantial absence of excessive back mixing leads to plug flow of liquid through the bed (Sonnenfeld et al., 2007). Practically, this means that in the expanded bed the particles have moved apart from each other, but are still in the same relative three dimensional position and there is no contact between them.

Accordingly, the bioreactor concept employed in the present invention makes use of the ability of an expanded column of liquid medium to support a particulate bed in floatation provided that certain criteria are met. For example, that the particles must be buoyant at moderate superficial velocities by virtue of a careful choice of their density and exposed surface area to the flowing medium. If the balance is suitable then form drag equals the gravitational force for each particle under the chosen conditions and the particle remains suspended with negligible movement. If this condition is met it becomes possible to support a bed of particles in a volume typically three times the resting (packed) volume. The position of each particle is relatively stationary with respect to its nearest neighbours and collision frequencies are low. In addition, it is preferable that the particle properties must satisfy biological conditions that provide a suitable scaffold for cell attachment and proliferation. The resultant bioreactor system operates under plug flow at moderate fluid shear. Such conditions will be attractive to cell and tissue growth whilst minimising diffusion limitations. Once the cell growth has been established and matrix produced the expansion will be stopped and the cells allowed to settle into a compact three dimensional bed, mimicking the complex structure of the required tissue. The result should be a tissue construct where the cells at the core will be proliferating at approximately the same rate as those on the surface. As a general rule, the above conditions will be met when the flow of culture media provides an expanded height of the bed of particles between 0.75 and 4.0 times, and more preferably between 1.5 and about 3.5 times, the height of the initial packed bed, and preferably an expanded bed which is about three times the height of the initial packed bed.

The work disclosed herein concerned three main areas of work: developing an expanded bed bioreactor; preparing suitable particulate materials to support cell and tissue growth in the reactor; and using the system to culture orthopaedic tissues using the example of ovine meniscal cartilage tissue.

### Development of an Expanded Bed Bioreactor

In the experiment disclosed herein, the development and use of several prototype columns resulted in the choice of the BioRad Econo Column Range (1.5 cm ID×15 cm or 20 cm length) combined with 1.6 ID tubing (silicone with Norprene at pump heads) and using a Watson Marlow 205 U multi-head pump. A 500 ml reservoir was used to serve all of the columns with media. This has inlet/outlets for each column stream and also a large bore gas inlet (0.2 μm filter) coiled into the media to permit passive diffusion of incubator air (95% air/5% CO₂).

In tandem with the development of the expanded bed column is the choice of support matrix. Preliminary work to prove the expanded bed reactor concept was based upon two considerations: firstly to achieve a stable expanded bed and secondly to achieve maintenance of ovine meniscal chondrocyte (OMC) culture. Two matrix types were identified as being potentially beneficial. Streamline DEAE (Amersham Pharmacia) is the matrix of choice for obtaining an expanded bed, since it had been specifically designed in graduated sizes and densities to be optimal for achieving stable expansion. A second matrix was identified, Cytodex (Amersham Pharmacia), which is a microcarrier of variable size and density and is optimised for cell attachment and growth. It is capable of fluidisation and stirred vessel culture, but has not been specifically used in expanded beds.

The main outcomes of this section of work are as follows: design and operation of a system capable of providing an expanded bed environment for culture was established. The bioreactor system was modified to ensure that the OMC were cultured within an area of expansion and not in a packed or fluidised bed regime. This reduces the potential for shear and detrimental inter-particle collisions. The theoretical physical parameters of the bioreactor have been established and methods for their calculation obtained (i.e. the drag force on spheres, terminal velocities, Reynolds numbers and prediction of bed expansion). This provides a means of characterising the system during operation and will lead to the development of design and modelling approaches for this reactor. Similarly, the key biological parameters have been identified and calculated. This is a relatively sparse area of research and thus the information obtained here provides a model for supporting cell and tissue culture within the bioreactor system. In this work, we showed that it was possible to culture the ovine meniscal chondrocytes over periods of up to one week on Cytodex beads. This provides evidence of the suitability of the EB system for the culture of cells.

Other aspects that were studied include: comparison of the EB system with other bioreactors (stirred, packed bed), causes of expanded bed instability were addressed and three existing methods of attaining bed stability in EBA were highlighted and applied to this system, preliminary investigation of an alternative suitable matrix to allow cell growth and stable expansion, column design and preliminary expansion experiments, mathematical analysis of expansion characteristics, refinements to expanded bed system, and finally, investigation of the final compression phase of reactor (see below).

### Preparation of Expanded Bed Materials

An important element of the EBR system is the development of the biodegradable matrix for attachment of cells. This has enormous implications for both the proliferation of the cells and the ability of achieving stable expansion. Whilst Cytodex, which was used in the preliminary work is commercially available to culture cells upon, this matrix does not fully biodegrade. A number of design criteria were identified for the bioresorbable matrix. This can be split into two categories: the biological properties and the physical properties. In the first instance the beads must allow the cells used to produce a particular bioengineered product (e.g. ovine meniscal chondrocytes) to attach, proliferate and to develop the correct phenotype. A key outcome was that the beads should support cell growth such that cells would produce their own extracellular matrix components. The physical properties included biodegradability, surface chemistry modifications to enhance cell attachment, and the correct physical properties to allow a stable expanded bed to form. A large number of beads were tested to screen for the above properties.

In summary, it was identified that an inter-relationship between surface chemistry, topography, and structure exists in order to develop the optimum beads. Surface chemistry ranges from homogeneous to heterogeneous, i.e. charged, biochemical cues, biomimetic surface (RGD). Topography varied from smooth to rough. Structure/shape varies with specific surface area. In this phase of work, screening of the candidate particles was carried out in stirred Techne flasks using dermal fibroblasts. The main particles that were studied in detailed were: natural particles, i.e. collagens and chitosans, synthetic particles and ceramics. Chitosan microspheres (ALVITO), were obtained as a range of particle sizes: soft porous microspheres (1-2 mm diameter, pore size 50-150 um); hard porous microspheres (100-200 um diameter, pore size 100 um); and soft solid microspheres (0.3 mm diameter, pore size 50-150 um). Synthetic particles of PLGA (75/25) and PEG-PLLA were tested. In addition, a variety of surface treatments were attempted to improve cell attachment. Finally, inorganic spheres of tricalcium phosphate (300 μm, pore size<0.5 um) were tested. The cells attached well to the ceramic spheres but were not suspended easily by stirring leading to poorer cell densities (as measured by total DNA analysis).

The main conclusions from this work were that chitosan-based microspheres performed better than PLGA microspheres, but do not perform well with collagen or ceramic-based microspheres. PEG-PLLA microspheres did not readily aggregate, but cell morphology was poor. It was found that macrostructure of the particles played an important role in 3D particulate culturing. In general increasing the particle surface area resulted in an increase in cell attachment. Collagen-based materials were superior for cell morphology compared to other particles. Cell spreading decreased in the following order: collagens<calcium phosphate<PLGA.

These particles studied here are not the only ones that could be used in an expanded bed bioreactor. Alternative particles are suggested above.

### Expanded Bed Culture of Meniscal Tissue

The optimum reactor configuration developed as described above was tested in detail to evaluate its ability to support OMC growth and to encourage the cells to produce native matrix molecules. In general, the cells were cultured in an “expansion” mode where the mass transfer limitations were minimised, then at a later time during the culture the flow was stopped allowing the bed of scaffold particles to settle, and then the flow was reversed culturing the tissue construct in a “compressed” mode.

A significant part of this work was the evaluation of the effect of both ascorbate (ASP) or growth factors on OMC proliferation and matrix formation (synthesis) in two bioreactor systems. Namely, rod-stirred flasks and the expanded bed bioreactor system. The effects of ASP treatment on Cytodex 1 and 3 bioreactor cultures were studied during both expansion and compression phases. 100 uM ASP was added after 24 h seeding and the culture was changed to packed bed after 14 days. Data showed an increase in OMC proliferation in bioreactor cultures treated with ASP. Proliferation was also higher in the packed bed than in the expansion phase of culture. Collagen type I and II expression was detected in bioreactor cultures and was not affected by ASP treatment. In contrast, stirred-flask cultures (Techne) showed an upregulation of collagen type II in the presence of 100 μM ASP.

The effects of growth factor treatment (PDGF-AB at 10 ng/ml and TGF-β1 at 1 ng/ml) on OMC growth on both Cytodex 1 and 3 were studied during expansion and compression phases. 200 uM ASP was added after 24 h seeding to all bioreactor cultures. Growth factors were also added after 24 h and the culture was changed to packed bed after only 7 days due to excessive aggregation of microcarriers in the PDGF-AB treated Cytodex 3 culture. After 7 days in packed bed, an agglomerate of Cytodex 3 microcarriers had formed in this culture. On removal from the bioreactor a small proportion of the microcarriers detached and were analysed separately from the agglomerate. Data showed a high number of viable cells in the PDGF-AB treated Cytodex 3 culture on day 14 and the expression of GAG and collagen type I was significantly higher than in any other culture. Treatment with TGF-β1 caused an increase in OMC proliferation but had no effect on matrix production, and no aggregation of microcarriers was seen in these cultures.

Overall conclusions from bioreactor experiments were that the optimal microcarrier type and culture conditions used are dependent on the cell type and the culture system used. In the bioreactor system, compression of the particles acts to increase cell proliferation and enhances matrix production. A combination of ASP and PDGF-AB had significant effects on OMC Cytodex 3 bioreactor cultures.

### SUMMARY

The work described herein demonstrates the significant developments that have been made using the expanded bed bioreactor of the present invention to enhance bioreactor culturing systems for tissue engineering. This work can be divided into three main categories of experiment concerning the expanded bed, the particles and the conditions for suspended particulate culturing.

### Expanded Bed

1. Development of an expanded bed bioreactor for culturing mammalian cells and tissue on microcarrier beads at a variety of operating scale from laboratory-scale to manufacturing.
2. Establishment of a protocol for stable bed expansion.
3. Identification of the influence of growth factors, ascorbate and culture requirements for matrix production in chondrocytes.
4. Introduction of expanded bed compression leading to high cell density and collagen production.
5. Development of parallel-operated bioreactor systems for reactor optimisation.
6. Establishment of enhanced seeding protocols using Techne flasks to improve reactor performance.
7. Evaluation of enhanced analysis methods for fibrochondrocyte proliferation.

### Particles

1. Detailed comparison of Cytodex 1, 2, and 3 microcarriers to support OMC (ovine meniscal chondrocyte) culture in particulate bioreactor systems.
2. Design, screening and development of biodegradable microspheres for use as tissue engineering scaffolds in bioreactor systems.
3. Identification of other shapes and structures of resorbable materials for culturing in 3D particulate culture systems.

### EXPERIMENTAL EXAMPLES

### Study 1—Proof of Principle Using Meniscal Chondrocytes

The development of a bioreactor system for culturing progenitor cells or cells for tissue engineering is heavily influenced by the fact that the cells are anchorage dependent and must be attached to a matrix. The remit is also dictated by the desire to achieve the novel concept of expanded bed culture. Traditional bioreactors have immobilised cells using a variety of cell attachment systems. Immobilisation methods such as using ballotini beads (Brown et al., 1988), hollow fibres (Tharakan and Chau, 1986) and encapsulation (Lee and Palsson, 1990) differ from tissue engineering immobilisation because in the latter the immobilisation method is usually part of the product.

Perfusion culture is the steady flow of media through a cell population. Cells are usually retained within a chamber, here a column. Perfusion removes the feed or famine cycles that usually occur in static and batch cell cultures and the continual supply of fresh nutrient allows the culture of potentially higher final cell densities. A main advantage of a perfusion bioreactor is that it provides enhanced delivery of nutrients throughout the entire construct by mitigating both external and internal diffusional limitations as fresh medium is not only delivered to the surface of the construct, but also throughout the internal structure of the construct (Bancroft et al., 2003). In this novel perfusion system, the whole column of microcarriers is loaded with cells forming a suspension that represents a substrate maintained in suspension during the expansion phase and is later switched into a close packed column in the compression phase.

The aims of this study were to evaluate a novel expanded/packed perfusion bed bioreactor system, as described above, using as an example the culture of ovine meniscal chondrocytes.

### Materials and Methods

### Cell Isolation and Seeding

Ovine meniscal fibrochondrocytes (OMC) were isolated using a method adapted from Kuettner et al (1982). Briefly, menisci were aseptically dissected from ovine hind limbs obtained within 24 h of slaughter (Graystones Ltd., Hull, UK). Isolated menisci were immersed in phosphate buffered saline (PBS; Sigma, Poole, UK) containing 0.25% (v/v) gentamycin (Sigma), excess adipose tissue removed and each menisci was then cut into small fragments. The tissue fragments were incubated with 10 ml/g of 0.1% (w/v) pronase-E (VWR International Ltd., Lutterworth, UK) for 3 h at 37° C., with constant mixing. After washing with PBS, tissue fragments were then incubated with 10 ml/g of 0.2% (w/v) Worthington's collagenase type 2 (Lorne Laboratories Ltd., Twyford, UK) overnight at 37° C., with constant mixing. Digested tissue was filtered through 70 um cell strainers and the cells pelleted by centrifugation at 1000 rpm for 10 min. Isolated OMCs were suspended in Dulbecco's modified Eagles medium (DMEM; Sigma) supplemented with 10% foetal calf serum (FCS; Helena BioSciences Europe, Sunderland, UK), 2 mM L-glutamine (Sigma), 50 IU/ml penicillin, 50 ug/ml streptomycin (Sigma) and 1% (v/v) non-essential amino acids (NEAA; Sigma) (termed complete media; CM) and seeded at a density of 3×10⁴/cm² (termed passage 1; P1). Media was changed after 48 h and the cells fed twice weekly thereafter. Confluent OMC cultures were then harvested following treatment with trypsin-EDTA (Sigma), and seeded into roller bottles (Fisher Scientific UK Ltd., Loughborough, UK), termed passage 2; P2. For all experiments confluent cultures of OMC were used at P2-3.

### Preparation of Microcarrier Cultures

Cytodex, 1, and 3 (Amersham BioSciences UK Ltd, Little Chalfont, UK) were prepared according to manufacturer's instructions. Prior to use, the microcarriers were rinsed twice and resuspended in CM. Seeding of OMCs on microcarriers was performed by transferring the microcarriers to siliconised 500 mL rod-stirred culture flasks (Techne Ltd, Cambridge, UK) and OMCs while maintaining the optimal ratio of 12×10⁶ OMCs/300 mg Cytodex in a total volume of 20 ml CM at 10 rpm overnight. After seeding, microcarriers were transferred into each bioreactor flow chambers. The volume into each rod-stirred culture flask was then increased to 100 ml with CM and cultures maintained at 37° C. for 14 days, with constant stirring at 20 rpm. These rod-stirred cultures were used as controls on each bioreactor experiment.

### Bioreactor Design and Assembly

The reactor consists of a cylindrical glass (Econo Column, BioRad, UK) flow chambers or column (1.5 cm I.D×15 cm length). Each column drew media from a 50 mL bottle reservoir (Biochem-Valve Omnifit, Cambridge, UK). Medium is continuously circulated from bottom to top of each column in the expansion phase and from top to bottom in the compression phase. Medium in each reservoir was half replenished every two days. The entire flow perfusion system was connected with silicone tubing (1.6 I.D, Masterflex, Cole-Palmer), that has a high permeability to both carbon dioxide and oxygen ensuring adequate equilibration with the surrounding incubator air. Due to the low mechanical durability of silicone tubing, norprene tubing (Pharmed, Fisher Scientific, UK), a more rigid tubing was used for a short segment of the circuit within the pump. The pump driving the force is a 12-channel peristaltic pump (Watson Marlow 205 U). This pump gives accurate flow rates in the range of 0.1 to 5 mL/min with the tubing size used in our system (1.6 I.D.). All bioreactor components, columns and reservoirs, were siliconised (SigmaCote, Sigma, UK) before sterilised by autoclaving.

### Setting Up the Bioreactor System

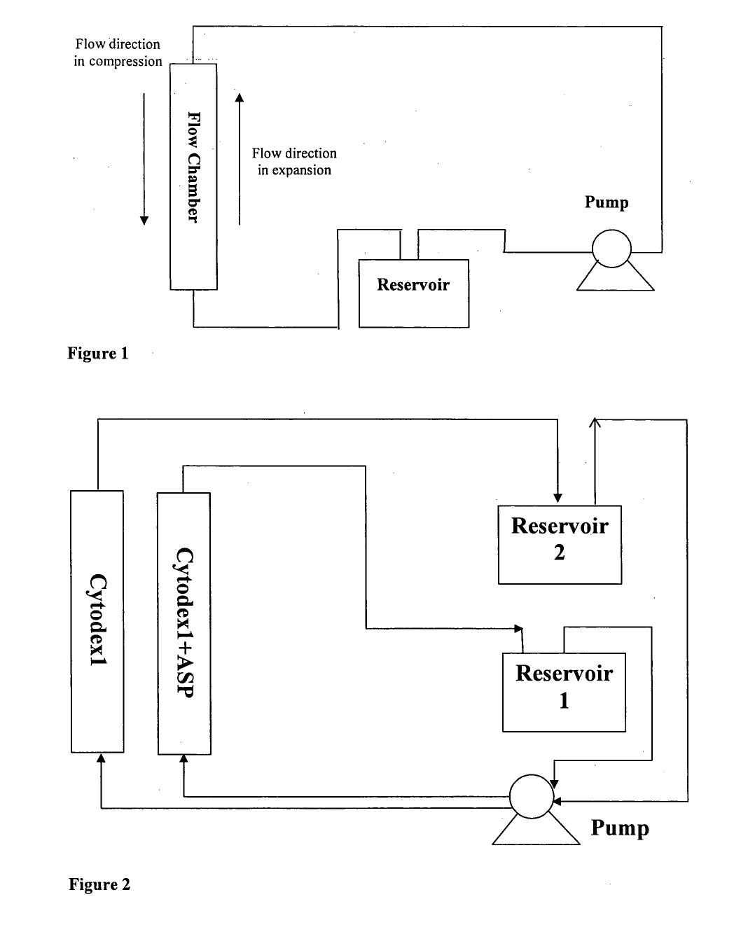

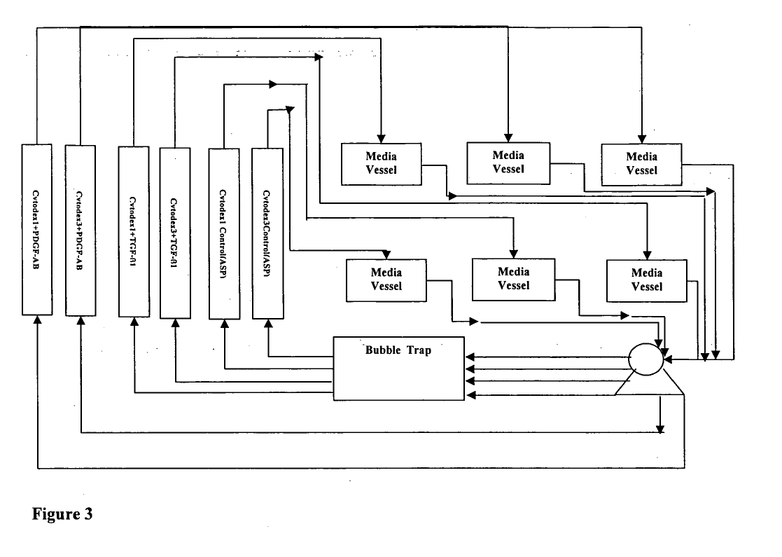

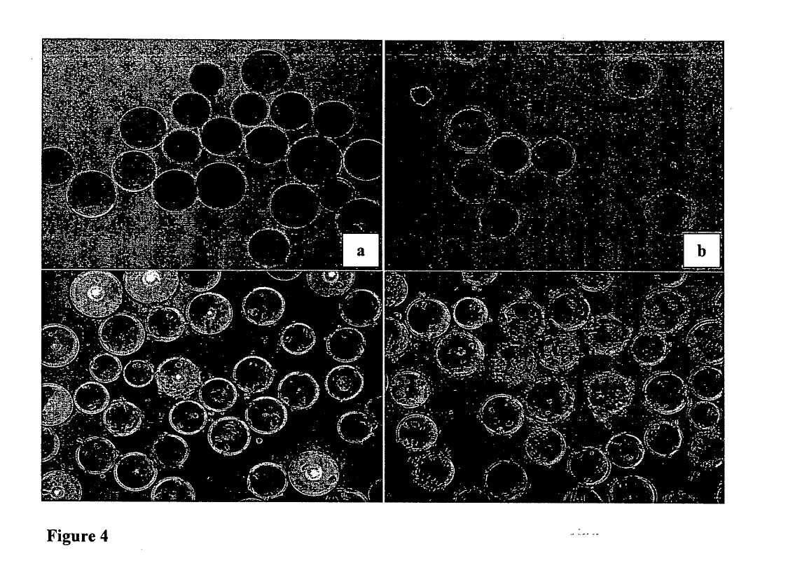

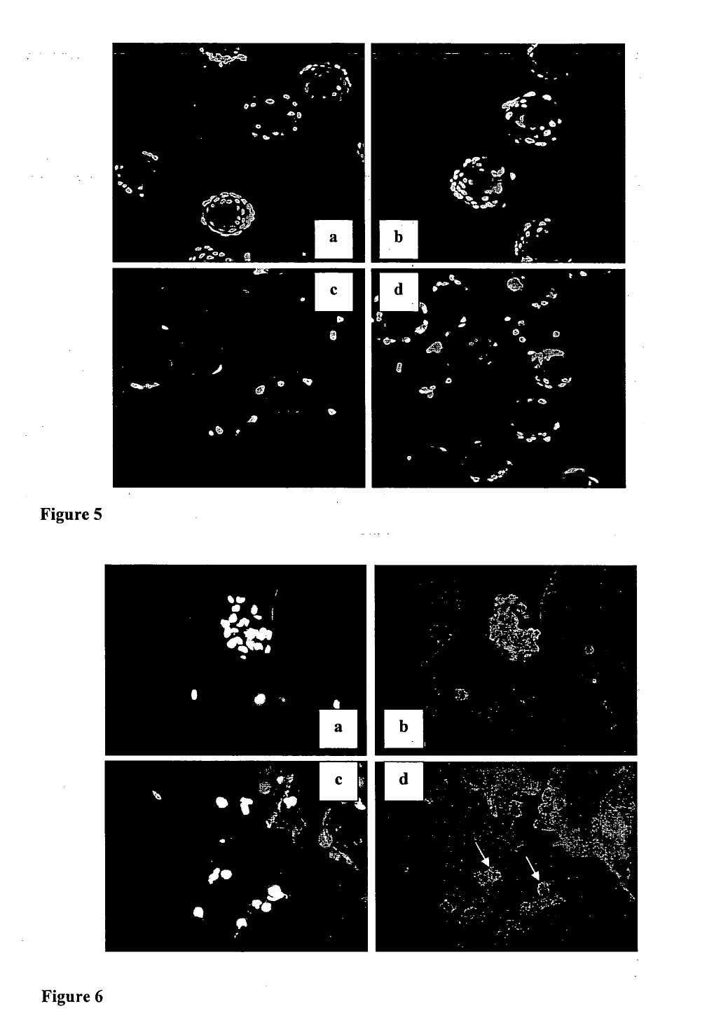

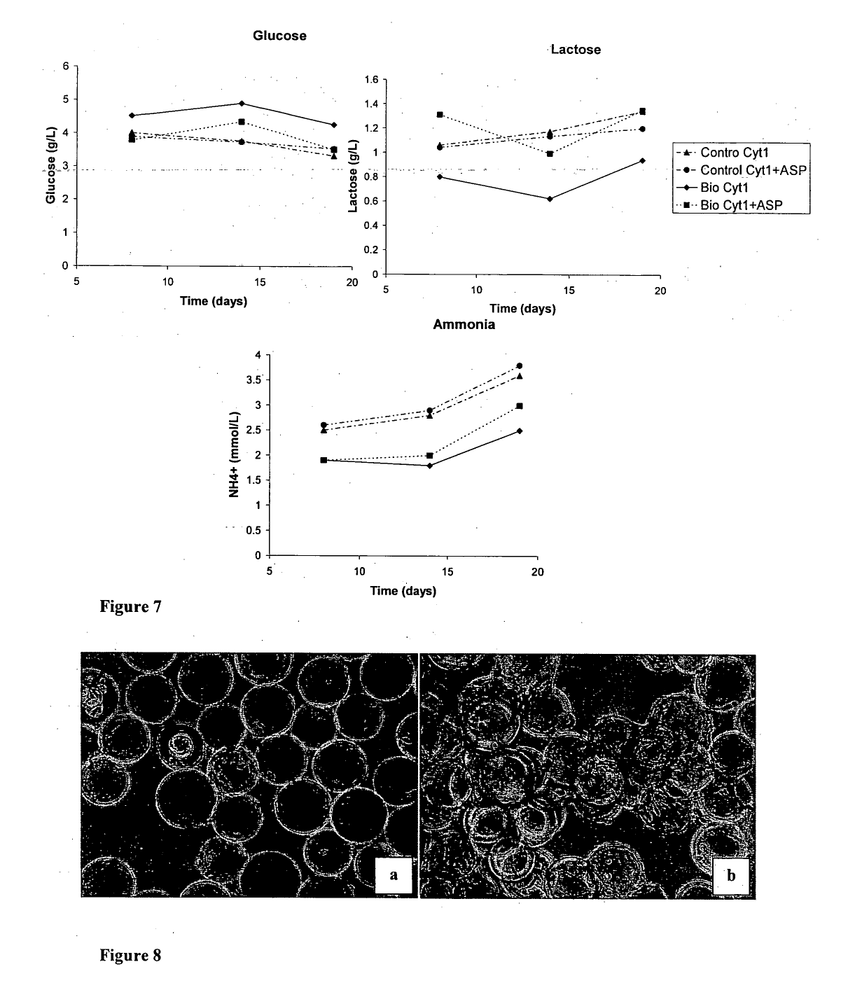

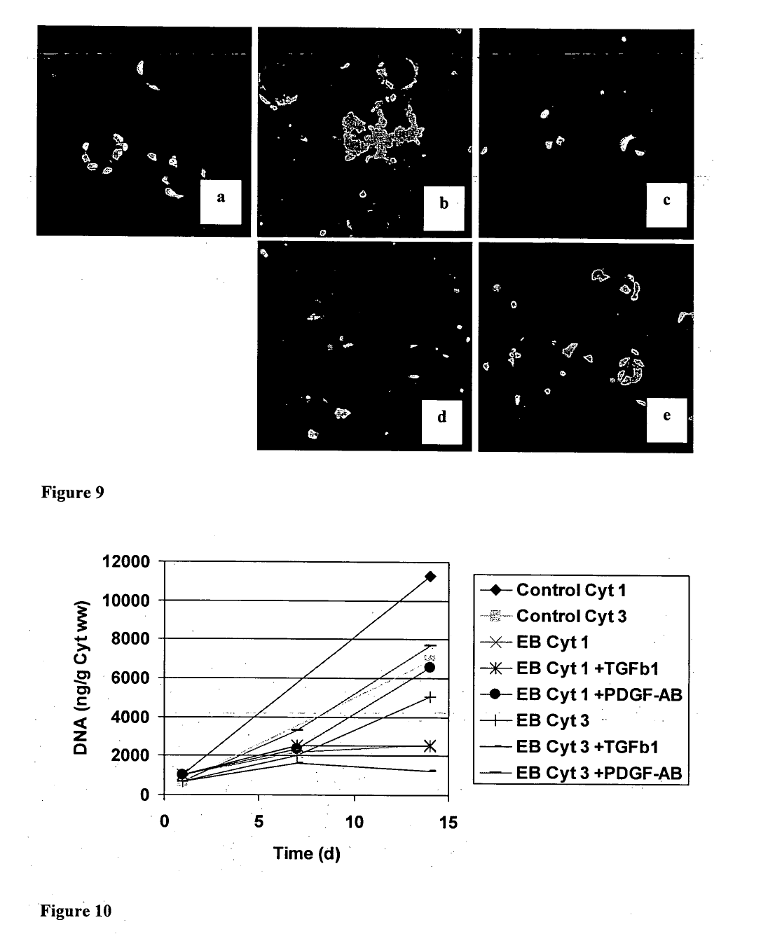

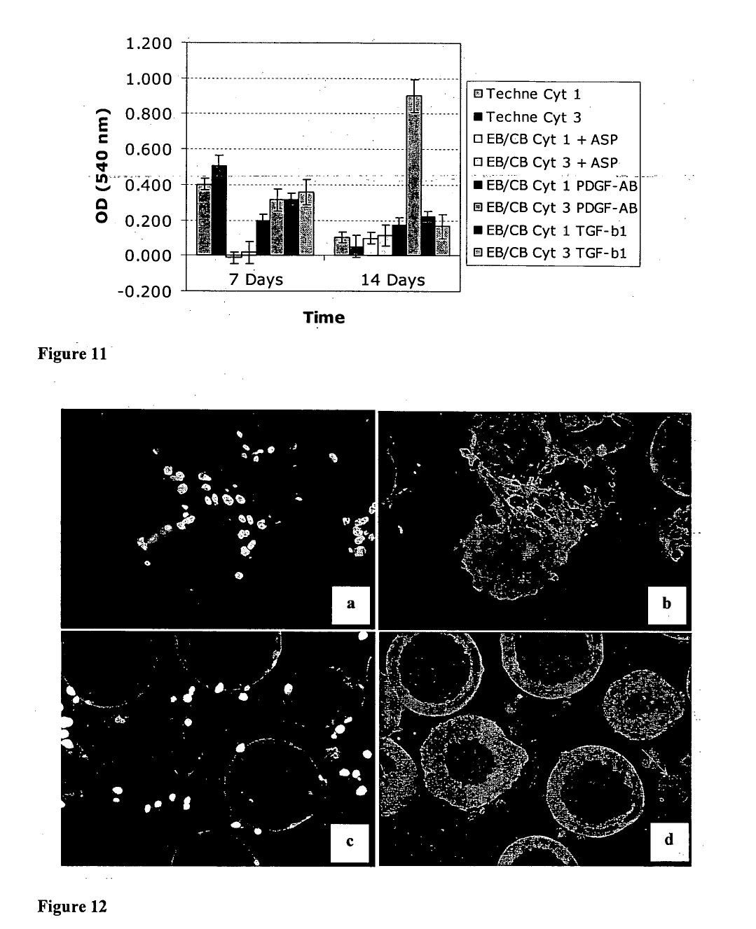

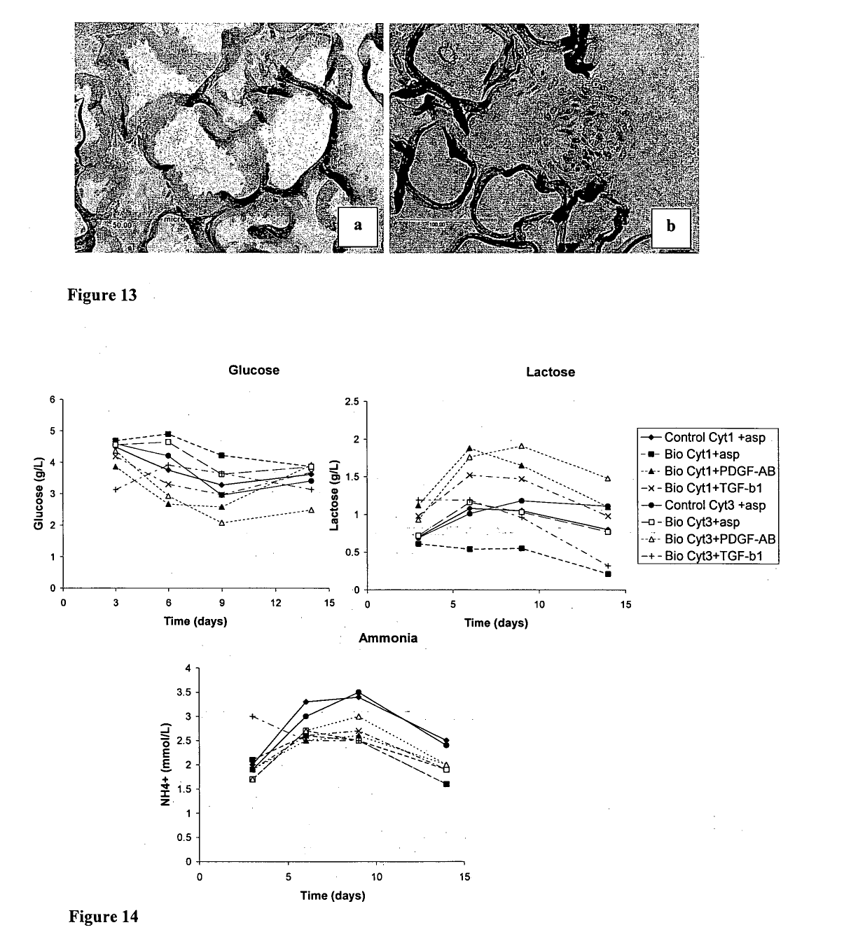

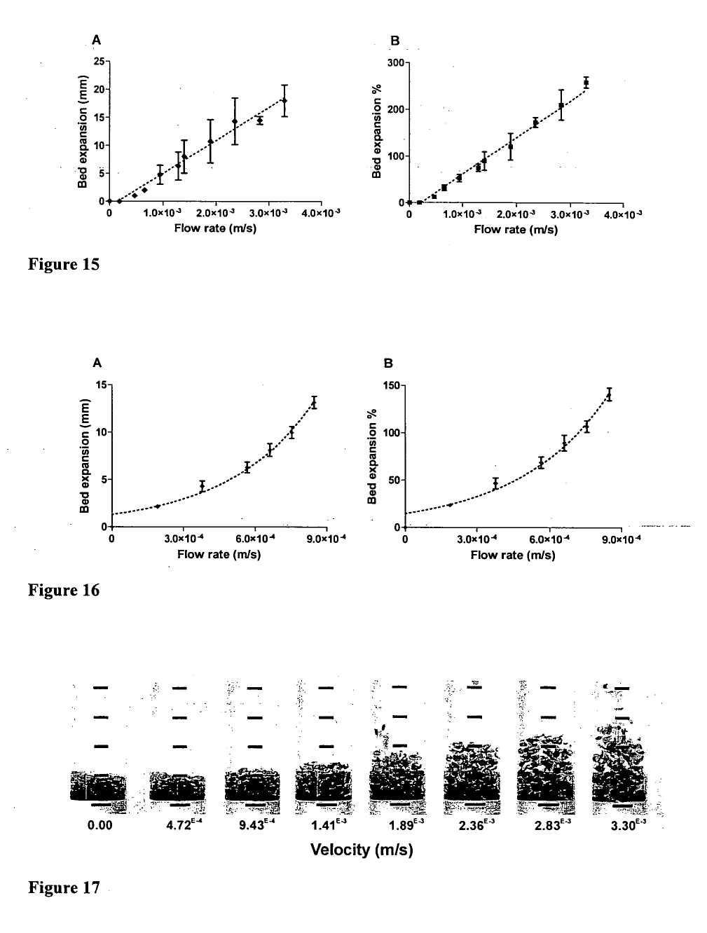

The complete bioreactor system was aseptically assembled in a laminar flow hood. Each flow chamber had its own media vessel and tubing within each channel of the peristaltic pump (FIG. 1). To be sure that air bubbles are completely remove from the system, a small volume of CM was added into each column (6 mL), this volume filled the tubing from the bottom of the chamber to the reservoir. After this, a clip was placed on the tubing below the column, ensuring that the column did not empty when seeded microcarriers were added into the columns.

The selected volume of microcarriers was added and a final volume of complete media to fill the chamber completely. The volume of microcarriers added into each column maintains the optimal ratio of 12×10⁶ OMCs/300 mg Cytodex kept in the control rod-stirred flasks. Finally, 50 mL of CM was placed into each reservoir.

The whole bioreactor system was then transferred to the incubator and after connecting each bioreactor to the pump, the clip on each one was removed and the pump started. The final flow rate (0.5 mL/min) was the same on either expansion or compression, the only difference was on the flow direction.

### Analysis Methods

Visualisation of Cells. Haematoxylin Staining

Microcarrier suspension was transferred to a non-tissue culture treated 6-well plate and washed twice with PBS. Cells were then fixed in 50% (v/v) methanol PBS for 10 min at followed by cold 70% (v/v) methanol PBS for 10 min. After removing the fixative, diluted haematoxylin solution (2-3 drops/10 mL distilled water) was added overnight at room temperature. Suspensions were then rinsed in tap water and visualised by light microscopy.

Cell Viability. Live/Dead Staining

Analysis of viability was performed at Day 14 in both experiments. From each Control and Bioreactor Column, 2 mL culture was removed and placed into a 15 mL conical tube, then washed gently with PBS. The supernatant was aspirated and 2 mL of live-dead stain solution (20 uL EthD-1 stock solution+5 uL calcein AM stock solution per 10 mL PBS, Live/Dead viability kit, Molecular Probes, USA) was added. Tubes were wrapped with foil and incubated at room temperature for 30-45 min. The samples were transferred to 60 mm petri dishes for observation using a confocal microscope (Zeiss LSM 510).

### DNA Quantification Assay

Samples of cells and microcarriers were transferred to 1.5 mL tubes and rinsed twice with PBS (Sigma) then pelleted at 2000 rpm for 2 min. The samples were resuspended in molecular biology grade water (CLP Ltd, Northampton, UK) and subjected to three cycles of freezing at −80° C. and thawing at 37° C. to ensure release of all DNA. Analysis of DNA content was performed using a PicoGreen®dsDNA quantitation kit (Invitrogen Ltd, Paisley, UK). Dilutions of samples were prepared in TE at 1:2-1:10, and dsDNA standards were prepared in TE at 0-1000 ng/mL. 100 uL aliquots of each were transferred to 96-well assay plates with each dilution assayed in triplicate. 100 uL/well PicoGreen® reagent was added 2-5 min prior to reading the samples using a fluorescence microplate reader (Fluoroskan Ascent; Labsystems, Finland) at Ex 420 nm, Em 520 nm. A standard curve was performed using cells alone by aliquoting 100 μL OMCs at various concentrations directly into 1.5 ml tubes. Each cell dilution was performed in duplicate. Cells were rinsed twice in PBS then lysed and analysed for DNA content as described above.

### Colourimetric Assay

The MTT assay was adapted from that of Mosmann (1983). Briefly, 100 uL of a 0.5 mg/mL solution of MTT (Sigma) was added to each well containing cells and microcarriers, and the cultures incubated for 4 h at 37° C. Microcarriers were then collected into a 70 um cell strainer (BD Biosciences, Oxford, UK) and 1 mL of dimethyl sulfoxide (DMSO; Sigma) added to solubilise the formazan product. Subsequently, 100 μL aliquots were transferred to 96-well plates and the absorbance was measured at 540 nm using a microplate reader (Dynex MRX Revelation; Dynex Technologies Ltd, Worthing, UK).

### Immunohistochemical Analysis

Immunohistochemistry for type I and II collagen was using a modification of a previously described protocol (Gronthos et al., 1997). Microcarrier suspension was washed twice in PBS and pelleted. The pellet was embedded into a ‘well’ of OCT fluid and snap frozen in liquid nitrogen then stored at −80° C. 10 μm sections were cut using a cryostat and air dried for 60 min prior to fixing in cold acetone for 5-10 min. Sections were fixed for 20 min in serum-free protein block (Dakocytomation Ltd., Cambridge, UK) then incubated for 60 min with either anti-type I antibody (1:20 dilution; Biogenesis), anti-type II collagen antibody (1:25 dilution; Abcam) or an equivalent concentration of rabbit IgG as an isotype-matched control (Sigma). After washing three times in PBS, sections were incubated with a biotinylated anti-rabbit antibody (2 ug/mL; Dakocytomation Ltd.) for 45 min. After further washing, a streptavidin-FITC conjugate (15 μg/ml; Dakocytomation Ltd.) was added for 20 min. Finally, sections were washed extensively in PBS and counterstained using 1 ug/mL 4,6-diamidino-2-phenylindone (DAPI) at 1:1000 dilution for 5 min then mounted using fluorescent mounting media and observed using a fluorescence microscope.

### Histological Analysis

A microcarrier suspension (2 mL) was removed from each culture system and washed twice with PBS (Sigma) before fixation by immersion in a solution of 4% formaldehyde. Microcarriers were then collected into a 70 um cell strainer (BD Biosciences, Oxford, UK) to minimise loss of microcarriers during preparation of each sample. After that, samples were dehydrated stepwise (70%, 95%, 100%) to 100% ethanol and incubated at least 8 h in a solution (1:1) of 100% EtOH: 2-hydroxyethylmethacrylate (GMA, Technovit 7100, Kulzer, Germany). Sample were kept in vacuum for 20 min to ensure optimal impregnation before immersion in embedded solution of GMA with hardener (Technovit 7100 kit, Kulzer, Germany) in a teflon mould. After polymerisation, 6 μm sections were obtained in a rotary microtome (Leica RM 2145, Leica, Germany). Embedded samples were stained using one of the following procedures: haematoxylin and eosin, safranin-O/fast green for GAG production and Masson-Goldner Trichrome stain for collagen production.

### Measurement of Cell Metabolites

At each time point, 5 mL of medium was taken from each reservoir and rod-stirred flasks and stored at −80° C. On the day of analysis, samples were thawed completely and 1 mL of each specimen was transfer into 1.5 mL tubes for analysis. Na⁺, K⁺, Ca²⁺, NH₄⁺, glucose, glutamate, glutamine, lactate concentrations and pH were measured using a BioProfile 400 Analyser (Nova Biomedical, Waltham, USA).

### Statistical Analysis

ANOVA one-way analysis of variance between groups with Tukey post test was carried out using PRISM software (GraphPad Software, Inc., San Diego, Calif., USA).

### Results

In this study, we have used a novel, expanded bed bioreactor system for the high cell density culture of meniscal chondrocytes.

In the first experiment, we set up the column to culture chondrocytes in an expansion phase, and have investigated the effects of adding ascorbate to the cells. OMCs were seeded onto Cytodex 1 microcarriers, and grown in expanded bed bioreactors with and without 100 μM ascorbate. The bioreactor cultures were kept expanded for 14 days after which, the flow direction was reversed and the compression phase was maintained for another 7 days (Day 21). A rod-stirred flask was used as a control and was supplemented with 100 mM ascorbate. Samples were removed from both bioreactors at regular intervals, 7, 14 and 21 days.

In the first experiment, the data showed an increase in OMC proliferation in bioreactor cultures treated with ASP compared to those without. Proliferation was also higher in the packed bed/compression phase than in the expansion phase of the culture. Collagen type I and II expression was detected in bioreactor cultures and was not affected by ASP treatment. In contrast, the rod-stirred cultures showed an upregulation of collagen type II in the presence of 100 uM ASP.

We also investigated the effects of specific chondrocyte growth factors, TGF-β1 and PDGF-AB, on cell culture on Cytodex 1 and 3 microcarriers in the expanded bed bioreactor. 200 uM ASP was added after 24 h seeding to all bioreactor cultures, and the growth factors were added after 24 h. The cells were cultured in expanded bed form for 7 days after which, the flow direction was reversed and the compression phase was maintained for another 7 days (Day 14). We investigated six experimental groups, with the results shown in the table below.

Collagen I and Collagen II Expression of OMCs grown on Cytodex 1 and Cytodex 3 in Control and Perfusion Bioreactor Cultures (14 days).

| | Collagen I | Collagen II |
|---|---|---|
| Control (Cyt 1 + asp) | ++ | + |
| Bio Cyt1 + TGF-β1 | + | + |
| Bio Cyt1 + PDGF-AB | + | + |
| Control (Cyt 3 + asp) | + | ++ |
| Bio Cyt3 + TGF-β1 | − | − |
| Bio Cyt3 + PDGF-AB | +++ | ++ |

In contrast to the study on the effects of ascorbate, the expanded bed bioreactor culture was changed to packed bed mode after only 7 days due to the high cell growth resulting in aggregation of the microcarriers in the PDGF-AB treated Cytodex 3 culture. Then, after a further 7 days in the packed bed configuration, an agglomerate of Cytodex 3 microcarriers had formed in this culture. The data showed a high number of viable cells in the PDGF-AB treated Cytodex 3 culture on day 14 and the expression of GAG and collagen type I was significantly higher than in any other culture. Thus collagen I expression was upregulated with PDGF-AB I the Cytodex 3 cultures. Treatment with TGF-β1 caused an increase in OMC proliferation, but had no effect on matrix production, and no aggregation of microcarriers was seen in these cultures. In the control cultures, Collagen II expression was upregulated in Cytodex 3 cultures compared to Cytodex 1 cultures

The expanded bed bioreactor offers the advantages of using particulate scaffolds to give uniform cell seeding on the microcarriers, and thus the production of a uniformly seeded bioreactor. Cells are not limited to the substrate surface, as in scaffolds. This reactor also can be operated as both an expanded bed to allow cell proliferation and uniform cell coverage of the microcarriers, and then after significant proliferation occurs, through compressing the bed the cells are in close contact that enhances cell communication and benefits matrix production. This work has also shown the benefits of adding specific growth factors into the media to enhance matrix production.

Our overall conclusions are that the use of an expanded bed bioreactor offers a flexible and enhanced culture system in which to cultivate cells on scaffolds. The expanded nature of the bed ensures that cells are well perfused with nutrients minimising concentration gradients. In the bioreactor system, compression acts to increase cell proliferation and enhance matrix production. A combination of ASP and PDGF-AB has significant effects on the culture of OMC on Cytodex 3 microcarriers.

### Study 2—Introduction

The work set out above focused on expansion of a particle bed using commercially available microcarrier systems for cell attachment (Cytodex®; Sigma-Aldrich, Gillingham UK). The work examined the potential for this system to be used to maintain populations of meniscal fibrochondrocytes and promote cartilage tissue formation. This study developed the initial success achieved with the microcarrier system to examine expansion of a larger carrier system (alginate/chitosan capsules) for cell encapsulation aimed to be a viable bioprocess for stem cell population expansion.

### Materials and Methods

### Cell Culture

### Isolation and Culture of Adult Human Bone Marrow Stromal Cells (HBMSC)

Adult human bone marrow samples were obtained from haematological normal patients undergoing routine elective hip replacement surgery. Only tissue that would have been discarded was used with the approval of the Southampton & South West Hampshire Local Research Ethics Committee. Primary cultures of bone marrow cells were established. Briefly, marrow cells were harvested using αMEM from trabecular bone marrow samples and pelleted by centrifugation at 500 g for 5 min at 4° C. The cell pellet was resuspended in 10 ml α-MEM and passaged through a 70 μm pore size nylon mesh cell strainer. Cell cultures were maintained in basal medium (α-MEM containing 10% FCS, penicillin 100 U/ml, streptomycin 100 μg/ml) at 37° C. in humidified air with 5% CO₂.

### Isolation and Culture of Human Fetal Femur-Derived Cells

Human fetal femurs were obtained and isolated following termination of pregnancy according to guidelines issued by the Polkinghome Report and with ethical approval from the Southampton & South West Hampshire Local Research Ethics Committee (LREC number; 296100). Primary cultures of HFF-DC cells were prepared as previously described and characterised. Briefly, fetal age was determined by measuring fetal foot length (range of 7.5-11 weeks post conception) and the femurs were placed in sterile 1× phosphate buffered saline (1×PBS) prior to removal of surrounding skeletal muscle. Femurs were dissected and cell isolation was achieved by a collagenase B digest. Cells were then placed into T25 flasks in 2 ml basal culture medium (α-MEM supplemented with 10% Fetal calf serum, penicillin, 100 U/ml and streptomycin, 100 U/ml) and changed weekly. Cells were maintained at 37° C. with 5% CO₂ and cultured for 7 days from explants before passage and scaffold seeding.

### Alginate/Chitosan Capsule Formation

Alginate/chitosan capsules were made by hand using a dropwise technique previously described. In summary, ultrapure alginate (NovaMatrix, Norway) (0.2 g) was added to 0.09 g sodium chloride and 0.3 g d-sodium hydrogen orthophosphate (210 mM) and dissolved in 5 ml distilled water to make a 4% (w/v) stock solution. Chitosan (3 g) was added to 1 g calcium chloride (50 mM), 3 ml acetic acid and 200 ml distilled water for a 1.5% (w/v) solution. A 1.5% (w/v) alginate solution was taken up in a 1 ml syringe and dispensed dropwise into wells of a six-well plate containing the chitosan solution using a 30 G needle. The droplets formed capsules approximately 1 mm in diameter which were left for 1 hr to gel. Capsules were removed from the chitosan solution and washed three times in sterile PBS solution.

### Cell Encapsulation in Alginate

Prior to encapsulation, human fetal femur-derived cells (HFF-DC) and human adult bone marrow stromal cells (HBMSC) were incubated with Vybrant CFDA SE Cell Tracer Kit, l Ethidium Homodimer-1 (Vybrant/EH-1) according to the manufacturer's instructions to label viable and necrotic cells respectively. Briefly, prior to encapsulation cells were trypsinized, centrifuged and incubated in basal culture medium (α-MEM/10% FCS, penicillin, 100 U/ml and streptomycin, 100 U/ml)) containing Vybrant/EH-1 at 37° C. for 1 hr. Samples were washed twice with basal culture medium, then bathed for a further 60 mins in basal culture medium only. An alginate (1.5%) solution was added to a cell pellet and vortexed to ensure thorough mixing and even distribution of cells throughout the alginate solution. Droplets of alginate encapsulated with cells (1 mm in size containing approximately 2×10⁴ cells) were dispensed onto the surface of the chitosan solution in a 6-well plate using a 1 ml syringe fitted with a 30 G needle. The capsules were left in the chitosan in a covered petri dish for 1 hr following self-assembly for the attachment of the chitosan shell to occur and were subsequently washed 3 times in tissue culture media.

### Characteristics of Expansion

### Bioreactor System Design

The bioreactor system was constructed using Omnifit chromatography columns (Bio-Chem Valve/Omnifit, Cambridge UK) connected to a fluid reservoir via Masterflex Platinum cured silicone tubing L/S14 (1.6 mm inner diameter) and the fluid flow driven by a peristaltic pump (Masterflex L/S digital drive with an L/S 8-channel pump head) with Masterflex two-stop pump Pharmed® tubing L/S14 (1.6 mm inner diameter; all supplied by Cole-Parmer Instrument Company Ltd., London UK). A schematic of this set up and an image of a set of running expanded bed columns can be seen in FIG. 1.

### Particle Expansion

Alginate/chitosan capsules (˜1 mm diameter) without cells were produced as previously described. Capsules were stained in a 25% (v/v) solution of Harris Haematoxylin in PBS for 30 mins and then washed 3 times in PBS. A total of 300 capsules or 50 mg of Cytodex 3 microcarriers were added to an Omnifit column (15 mm diameter, 100 mm in height, 100 μm PTFE frits with an adjustable end piece) as part of the bioreactor system previously described. PBS for alginate/chitosan capsules or DMEM media for Cytodex 3 microcarriers was used as the fluid within the system for this study. The flow rate was regulated via a peristaltic pump and increased in regular increments and bed height measured at each increase. This process was conducted 4 times for each particle type using different particle beds. Data collected is represented in FIGS. 16 and 17.

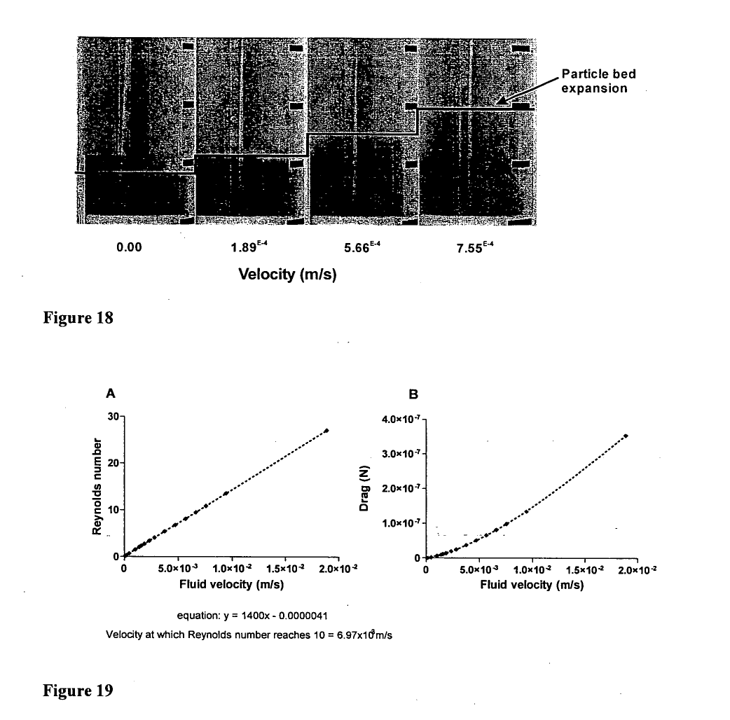

Representative images of the different particle beds at different stages of expansion were taken and have been arranged to give a visual representation of the occurring bed expansion (FIG. 17, 18). From bed expansion measurements for alginate/chitosan capsules (FIG. 16) it is clear that a particle bed consisting of these capsules follows typical expansion progression. There is an initial lag phase (up to the velocity of 4.72×10⁻⁴ m/s), due to unequal gravitational forces from the capsules against the fluid flow. A linear expansion follows with relation to increasing fluid velocities as the fluid forces match the buoyancy of the capsules (in the range of 4.72×10⁻⁴ to 2.36×10⁻³ m/s). This is clearly illustrated in the series of images shown in FIG. 16. The particle bed was reaching fluidisation point at 2.83×10⁻³ m/s which is evident from the images in FIG. 18. The expansion and fluidisation of the alginate/chitosan capsule particle bed is comparable to that formed from Cytodex microcarriers with the difference only in the velocities required to cause expansion. Similarities of lag and linear phases are clear in the data presented in FIGS. 16 and 17 with additional support provided from the images displayed in FIGS. 18 and 19.

### Shear Stress Calculations

As the alginate/chitosan capsule particle bed requires increased force to enable bed expansion, it is important to assess the potential local forces that may effect cellular growth. To determine the localised physical environmental conditions effecting the capsules, drag/shear forces acting on the capsules were determined using the equations 1, 2 and 3[5].

$$\begin{matrix}Re \\ euD\end{matrix} = \frac{ }{ } . \qquad Equation 1$$

Where:
Re=Reynolds number D=sphere diameter
e=fluid density μ=fluid dynamic viscosity
u=fluid velocity

$${C}_{D} = \frac{18.5}{{Re}^{0.6}} . \qquad Equation 2$$

Where:
C_{D}=drag coefficient
Re=Reynolds number

$$D = \frac{1}{2} {ρ}_{f} {C}_{D} {A}_{p} {v}_{f}^{2} . \qquad Equation 3$$

Where:
D=drag ρf=fluid density
C_{D}=drag coefficient Aₚ=sphere area (flat circle)
v_{f}=flow velocity

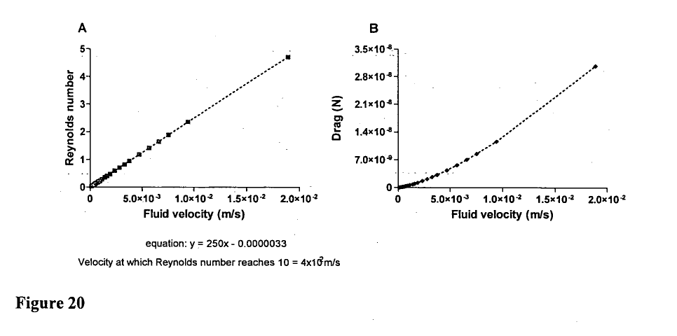

From calculating a range of Reynolds values, flow around the sphere was determined via Allen flow equations. As the Reynolds values were found to be below a value of 10 within the system, drag and shear were considered to be the same value. Calculated values for shear forces are shown in FIGS. 19 and 20.

Comparison of the two particle systems shows similarities in their associated trends for Reynolds values and drag levels. Difference in actual values can be addressed due to the particles variation in size and density. The most important point is that in the fluid velocity ranges that cause stable bed expansion (4.27×10⁻⁴ to 2.36×10⁻³ m/s for alginate/chitosan capsules and 1.89×10⁻⁴ to 8.49×10⁻⁴ m/s for Cytodex microcarriers) both produce low levels of drag acting on individual particles within the bed, a maximum of 1.93×10⁻⁸N and 3.99×10⁻¹⁰ N respectively.

### Cell Culture in Expansion

### Determination of the Effect of Encapsulated Cell Number

A total of four particle beds of 300 alginate/chitosan capsules (2 columns containing 1×10⁴ adult BMSC/capsule, 2 columns containing 3×10⁴ adult BMSC/capsule) were expanded between 80-100% of their original height with a media flow rate of 15 ml.min-1 (0.0014M.sec⁻¹) ±2 ml.min⁻¹ and cultured for a period of 7 days at 37° C. and 5% CO₂. These studies were run in order to determine an optimum encapsulation cell number. Comparison of cell encapsulation concentration showed a significant difference in DNA levels (4.2 & 4.0 μgml⁻¹ measured for 3×10⁴ cells/capsule compared to 0.7 & 1.0 μgml⁻¹ measured for 1×10⁴ cells/capsule; p<0.001) and an increase in mean alkaline phosphatase activity (4.7 & 4.4η mol PNPP-1 mg DNA⁻¹ for 3×10⁴ cells/capsule compared to 0η mol PNPP-1 mg DNA⁻¹ for both 1×10⁴ cells/capsule) after 7 day culture.

### 7 Day Cell Cultures

Four columns containing 300 capsules with 2×10⁴ fetal BMSC/capsule were expanded to 80-100% bed height as previously described and cultured for 7 days at 37° C. and 5% CO₂. The flow rate used to achieve bed expansion was maintained at 15 ml.min⁻¹ (0.0014M.sec⁻¹) ±2 ml.min⁻¹. This experiment was to judge the reliability of the bioreactor system, to observe the expanded bed over a period of time and assess the cell number encapsulated in the capsules. Observations indicated that the expanded bed height was maintained at a consistent level and the system proved reliable over a 7 day period. No significant difference was noted between static and expanded bed cultures over 7 days for both DNA levels (mean of ˜3 μg) or alkaline phosphatase activity (negligible levels).

Both static and expanded bed capsule cultures showed no staining for alcian blue and Sirius red. Cells were spread out across the capsule with small areas of cell clustering. In both sets of culture conditions there was very little immunoexpression of Typ-1 collagen. Weak staining of type 1 collagen was noted in some of the small clusters of cells in the alginate/chitosan capsules, but no obvious differences were portrayed between the two groups.

### Discussion

Characterisation of an alginate/chitosan capsule based expanded bed has shown that it is possible to use these particles to successfully generate stable expanded beds for cell cultures. Further to this, it can be added that by carefully selecting the particles for the expanded bed it would be possible conduct larger scale cell cultures. The only possible factor that would become important would be the drag/shear forces that would be generated around the individual particles. However, this could be beneficial in some future tissue engineering processes as increased shear and pressure would be beneficial for tissue growth. In addition, an alginate/chitosan capsule system cells are encapsulated within the alginate and protected by a chitosan shell so localised forces acting on the outside of the capsule may not directly affect the cells inside the capsules. It should be noted, however, that alginate encapsulation may have limitations with regard to mass transfer controlled not by the level of fluid flow but by the chitosan shell or alginate gel. These issues make the flexibility of this expanded bed bioreactor system even more desirable as it has the potential to be tailored to any specific requirements.

Using a cell encapsulation technique with alginate and chitosan, we were successful in generating a stable expanded bed which could maintain a viable population of human stromal cells over an extended culture period. This was demonstrated by 7 day cultures of human fetal and adult stromal cells showing good viability. Cell encapsulation numbers were determined at an optimum level of 20,000 cells per capsule as lower cell concentrations produced negligible DNA levels and 20,000 cell per capsule would provide a good starting point for cell expansion. Similarities were noted between static and expanded bed cultures for encapsulated HFF-DC in both DNA, alkaline phosphotase and histological analysis. This observation at this early stage should not necessarily be considered a failure. It is encouraging as it shows that cells are stable within the alginate/chitosan capsule and that the expansion has not detrimental affect on the cells. It is now increasingly important to extend cell culture periods within this system to determine the effects on the cell population. Ongoing work is examining human fetal femur derived cells, adult bone marrow stromal cells and STRO-1 isolated stromal progenitors cell growth and activity (with regard to differentiation) over a 21 day period. Current results still show a stable and robust system and indicate good cell viability over the extend time period. Some of this data is presented in Section 7 as it is still in progress.

### Conclusions

Good cell viability after encapsulation and incubation over 7 days for primary human cells was observed.

The use of a moderate cell number for encapsulation (2×10⁴ cells/capsule) provides an optimal encapsulation concentration. The system also provided a large enough population of cells to encourage cell proliferation.

The method left room for cell proliferation (limited volume as cells are encapsulated within the alginate), and also allows for small numbers of donor cells to be utilised effectively.

The bioreactor system has been demonstrated to be stable over 7 and 21 day periods and is robust enough and easily maintained for a 21 day incubation.

The similarities in DNA, ALP production and histological assessment between static and expanded bed culture conditions indicate that there is no detrimental effect of cells being grown under expanded bed conditions.

It is unlikely that stress related forces will inhibit cell growth as they are drag/shear have been calculated to be very low.

There is a potential for this system to be highly useful to both Mesenchymal Stem Cell and Embryonic Stem Cell research due to it's flexibility

Expansion followed by packing of the particle bed (stimulation for tissue generation) has been demonstrated.

The dosing of growth factors easily carried out within the system e.g. incorporation of slow release capsules for different growth factors within the particle bed.

Varying particle type/size to match conditions required for cell culture is possible, as is cell co-cultures using different size particles for cell separation within the column.

### Study 3—Introduction

An important feature of the expanded bed system is to provide long term stable cell culture conditions. A specific goal for the expanded bed bioreactor is the successful culture of human progenitor cells with the aim to provide large populations viable pluripotent cells. To meet these aims a series of studies using human fetal femur derived cells, adult bone marrow stromal cells and STRO-1 isolated stromal progenitor cells has been undertaken. The initial results from these studies is presented below.

### Extended Cultures in the Expanded Bed System

Four columns containing particle beds of 300 alginate/chitosan capsules with 2×10⁴ fetal BMSC/capsule were expanded by 80-100% original bed height as previously described, and cultured over 21 days at 37° C. and 5% CO₂. The flow rate used to achieve bed expansion was maintained at 15 ml.min⁻¹ (0.0014M.sec⁻¹) ±2 ml.min⁻¹.

This study aimed to determine the durability of the culture system over a prolonged culture period and to asses the cellular activity, proliferation and viability over the 21 day culture. Observations made concluded that the bioreactor system was highly robust and easily managed to maintain sterility. Media analysis showed no significant differences between static and expanded bed cultures or over time for measured levels, of glucose (˜0.9 g/L for day 7 and 0.7 g/L for both day 14 and 21) and lactate (between 0.06 and 0.007 g/L for all time points) in the media over the 21 day period. Calculations of cumulative glucose used over the 21 day culture period, showed no significant difference between static or expanded bed cultures at any point. A significant difference, however, was noted at day 21 for the calculated cumulative lactate production (static culture 5.42 g/L expanded bed culture 6.46 g/L; p<0.01). From non-dimensionalising the data through calculating the lactate to glucose ratio, no significant difference between the trends of static and expanded bed culture conditions were noted. Both suggested a trend of exponential growth as it appeared that the graphs were indicating they would plateau over time as glucose was used and lactate produced at constant levels within the systems.

In extended culture over a period of 21 days in the expanded bed bioreactor system, fetal femur derived cells encapsulated in alginate/chitosan capsules were observed adhered and viable as evidenced by extensive labelling of the cell viability marker Vybrant® and negligible EH-1 labelling, a marker for cell necrosis.

Both static and expanded bed capsule cultures showed minimal staining for alcian blue and Sirius red. Capsules stained for goldners trichrome (a measurement of collagen, osteoid formation and cell nuclei) demonstrated that cells were spread out across the capsule with small areas of cell clustering with negligible collagen or osteoid matrix deposition. Similarly both sets of culture conditions showed very little immunoexpression of Type-1 collagen.

### REFERENCES

All publications, patent and patent applications cited herein or filed with this application, including references filed as part of an Information Disclosure Statement are incorporated by reference in their entirety.
McCabe, W. L. and J. C. Smith, Unit Operations of Chemical Engineering. 1976, Tokyo: McGraw-Hill. 1028.
Chase, H A and Draeger, N M, Affinity purification of proteins using expanded beds, J Chromatogr. 24; 597(1-2), 129-45, 1992.
Sonnenfeld, A. and J. Thommes, Expanded bed adsorption for capture from crude solution, in Process Scale Bioseparations for the Biopharmaceutical Industry, A. A. Shukla, M. R. Etzel, and S. Gadam, Editors. 2007, Taylor & Francis Group: Boca Raton. p. 59-82.
J. F. Richardson and W. N. Zaki, Trans. Inst. Chem. Eng. 32 (1954), p. 35.
Yates, J. G., Fundamentals of Fluidized-bed Chemical Processes. 1983, London: Butterworths. 222.
Dong-Qiang Lin, Zhi-Jun Miao and Shan-Jing Yao (2006). Expansion and hydrodynamic properties of cellulose-stainless steel powder composite matrix for expanded bed adsorption, Journal of Chromatography A, 1107(1-2), 265-272.
Sweigart M A and Athanasiou K A. (2001) Toward tissue engineering of the knee meniscus. Tissue Engineering 7: 111-129.
Webber R J, Harris M G and Hough A J Jr. (1985) Cell culture of rabbit meniscal fibrochondrocytes: proliferative and synthetic response to growth factors and ascorbate. J. Orthop. Res. 3: 36-42.
Spindler K P, Mayes C E, Miller R R, Imro A K and Davidson J M. (1995) Regional mitogenic response of the meniscus to platelet-derived growth factor (PDGF-AB). J. Orthop. Res. 13: 201-207.
Collier S and Ghosh P. (1995) Effects of transforming growth factor beta on proteoglycan synthesis by cell and explant cultures derived from the knee joint meniscus. Osteoarthritis Cartilage 3: 127.
Bhargava M M, Attia E T, Murrell G A, Dolan M M, Warren R F and Hannafin J A. (1999) The effect of cytokines on the proliferation and migration of bovine meniscal cells. Am. J. Sports Med. 27: 636.
Kuettner, K. E., Pauli, B. U., Gall, G, Memoli, V A, and Schenk, R K, 1982, Synthesis of cartilage matrix by mammalian chondrocytes in vitro. 1. Isolation, culture characteristics, and morphology, J. Cell Biol. 93 (3):743-750.
Mosmann T (1983) Rapid colorimetric assay for cellular growth and survival; application to proliferation and cytotoxicity assay. J. Immunol. Methods 65: 55-63.
Hillsley, M. V., Frangos, J. A., “Bone tissue engineering: the role of interstitial fluid flow”, Biotech Bioeng, 43, 573, 1994.
Gronthos S, Stewart K, Graves S E, Hay S and Simmons P J, “Integrin expression and function on human osteoblast-like cells”, J. Bone Miner. Res. (1997) 12: 1189-1197.

1. A method of producing a tissue engineered product in a bioreactor which comprises:
(a) immobilising or encapsulating cells for forming the tissue engineered product on or in particles of a scaffold material;
(b) forming a packed bed of the particles and cells in a flow chamber of the bioreactor;
(c) passing cell culture media through the bed of particles in the flow chamber at a velocity sufficient to separate the particles to form an expanded bed under conditions tending towards plug flow and which substantially maintain the relative positions of the particles in the flow chamber;
(d) culturing the cells so that they proliferate on the particles to begin to form tissue elements; and
(e) forming the tissue elements on the particles to produce the tissue engineered product.

2. A method of culturing progenitor cells in a bioreactor which comprises:
(a) immobilising or encapsulating the progenitor cells on or in particles of a scaffold material;
(b) forming a packed bed of the particles and cells in a flow chamber of the bioreactor;
(c) passing cell culture media through the bed of particles in the flow chamber at a velocity sufficient to separate the particles to form an expanded bed under conditions tending towards plug flow and which substantially maintain the relative positions of the particles in the flow chamber;
(d) culturing the progenitor cells so that they proliferate on the particles; and
(e) isolating the cultured cells.

3. The method of claim 1, wherein the step (e) of forming the tissue engineered product comprising compressing the tissue elements on the particles to form the tissue engineered product and/or producing extracellular matrix.

4. The method of claim 2, wherein the step of compressing the tissue elements on the particles comprises stopping or reducing the flow of the culture media to allow the particles to settle under gravity.

5. The method of claim 2, wherein the step of compressing the tissue elements on the particles comprises reversing the flow of the culture media.

6. The method of claim 1, wherein the cells are mammalian primary cells, progenitor cells or genetically modified cells.

7. The method of claim 6, wherein the cells are progenitor cells and the method comprises culturing the progenitor cells in the expanded bed phase thereby maintaining pluripotency and/or multipotency and differentiating the cells in either the expanded bed and/or compressed bed phase.

8. The method of claim 7, wherein differentiating the progenitor cells comprises contacting the cells with growth factors.

9. The method of claim 1, wherein the tissue engineered product is a three dimensional tissue engineered product.

10. The method of claim 9, wherein the three dimensional tissue engineered product has a smallest dimension of at least 1 mm, of at least 2 mm, of at least 5 mm, or of at least 10 mm.

11. The method of claim 1, wherein the tissue engineered product is articular or meniscal cartilage, bone tissue, ligament, tendon, nerve cells, liver, pancreas, cardiac and vascular tissue, cornea, adipose tissue, genito-urinary tissue and/or dental tissue.

12. The method of claim 1, wherein the cells are meniscal chondrocytes, bone marrow stromal cells, mesenchymal stem cells, adult stem cells or embryonic stem cells.

13. The method of claim 1, wherein the tissue engineered product is formed from the co-culture of more than one cell type in the bioreactor.

14-26. (canceled)

27. The method of claim 1, wherein the particles are coated with a growth factor.

28. The method of claim 27, wherein the growth factor is fibronectin.

29-33. (canceled)

34. The method of claim 2, wherein the culture media comprises growth factors or other agents for differentiating progenitor cells to terminal cell types.

35. A tissue engineered product obtainable by the method of claim 1.

36. An apparatus for producing a tissue engineered product which comprises a generally cylindrical flow chamber having a fluid inlet and a fluid outlet, wherein the flow chamber of the bioreactor receives a bed of particles of a scaffold material on which cells are immobilised or encapsulated;
an inlet flow adapter in fluid communication with the fluid inlet of the bioreactor chamber for providing an even flow distribution;
an upper adapter in fluid communication with the fluid outlet to prevent cell loss and to allow flow reversal and bed compression; and
a pump for circulating culture media through the chamber via the flow adapters;
wherein in use (a) cell culture media is passed through the bed of particles in the bioreactor from the inlet to the outlet at a velocity sufficient to separate the particles to form an expanded bed under plug flow conditions which substantially maintains the relative positions of the particles, (b) the cells are cultured so that they proliferate on the particles, and optionally start to form tissue elements, and (c) the flow of culture media is stopped or reversed to compress the tissue elements on the particles to produce the tissue engineered product.
